(19) United States
(12) Patent Application Publication
(10) Pub. No.: US 2016/0213278 A1
(43) Pub. Date: Jul. 28, 2016

(54) Method for Detecting Arthritis and Cartilage Damage Using Magnetic Resonance Sequences

(71) Applicants: Kenneth L. Urish, Hershey, PA (US); Matthew G. Keffalas, Woburn, MA (US); Timothy J. Mosher, Elizabethtown, PA (US); David J. Miller, State College, PA (US)

(72) Inventors: Kenneth L. Urish, Hershey, PA (US); Matthew G. Keffalas, Woburn, MA (US); Timothy J. Mosher, Elizabethtown, PA (US); David J. Miller, State College, PA (US)

(21) Appl. No.: 14/971,782

(22) Filed: Dec. 16, 2015

Related U.S. Application Data
(63) Continuation-in-part of application No. 13/691,359, filed on Nov. 30, 2012, now abandoned.

(51) Int. Cl. A61B 5/055; A61B 5/00

(52) U.S. Cl. CPC A61B 5/055; A61B 5/4514; A61B 5/4528; A61B 5/7275

(57) ABSTRACT

A method for detecting symptomatic osteoarthritis in a human patient who is otherwise asymptomatic comprises: taking a plurality of magnetic resonance (MR) image signal features from MR sequences in a joint of the patient; submitting the MR image signal features to a classifier for performing a feature reduction for redundant or unnecessary features which are then eliminated; calculating a signal texture index (STI) value from the remaining image features; comparing that STI value against two population databases, one for individuals known to develop osteoarthritis and a second for individuals known not to develop osteoarthritis at a given time point; prognosticating from the STI value, a likelihood of the patient developing osteoarthritis; and treating the patient accordingly.

### CROSS-REFERENCE TO RELATED APPLICATION

This application is a continuation-in-part of application Ser. No. 13/691,359 filed on Nov. 30, 2012, which was a perfection of U.S. Provisional Application Ser. No. 61/629,876, filed on Nov. 30, 2011, both disclosures of which are fully incorporated by reference herein.

### BACKGROUND OF THE INVENTION

1. Field of the Invention

The present invention relates to using magnetic resonance imaging signal metrics, including but not limited to texture metrics, for prognosticating and diagnostic measurements of osteoarthritis (“OA”) and cartilage damage.

2. Relevant Art

Numerous references are known using MRI data to some extent in the same context as arthritis or cartilage. Such references include: Mangialaio et al PCT Application No. 2006/008183 (pertaining to the use of biomarkers for rheumatoid arthritis (or “RA”)); Licha et al. EP Application No. 1,931,391 (which employed optical imaging for RA); Wakitani et al. EP Application No. 2,128,615 (for detecting joint cartilage damage); Bukowski et al. PCT Application No. 2009/135219 (for detecting a predisposition for osteoarthritis); and Yen et al. Published U.S. Patent Application No. 2012/0128593 (for using MRI imaging in inflammation or infection detection). The most recent, Dam et al. U.S. Pat. No. 8,300,910 segmented cartilage and mentioned homogeneity but only in the context of damaged or suspect joints.

This invention represents an alternative to and improvement over the foregoing. With respect to Dam et al., this concept is distinct in our using a large number of several different MR signal measurements and combine same into a single measurement by selecting only the important, more critical measurements for assessing not only damaged joints but also those joints currently showing no sign of disease or damage for prognostication and imaging biomarkers.

Osteoarthritis (OA) is a common disease affecting approximately half of the population above the age of 55. Radiographs remain the standard imaging technique to assess OA disease progression. There is an interest in using Magnetic Resonance Imaging (MRI) to identify pre-radiographic changes in OA.

MRI has the capability to directly image cartilage. There have been preliminary applications of compositional MRI techniques to detect changes in water and proteoglycan content and anisotropy of collagen fibers [1-4] associated with early degradation, albeit with limited success [5]. The inclusion of cartilage T2 mappings in the Osteoarthritis Initiative (OAI) protocol was designed in part to develop such predictive capability. This more than 4-year longitudinal natural history study provides annual knee MRI examinations of almost 5000 subjects, and is a valuable resource for deriving image based biomarkers to identify individuals at risk for incident OA or rapid OA progression. The study provides the longitudinal data necessary for evaluation of cartilage T2 as a potential biomarker for predicting OA progression.

Normal T2 values of knee articular cartilage have a well-recognized pattern of signal variation, spatial signal distribution that changes with OA. The T2 values of articular cartilage are strongly dependent on the orientation of the type II collagen matrix with respect to orientation of the applied magnetic field BO (anisotropy) [2, 6], and regional differences in cartilage water content [2, 5, 7]. In normal cartilage, regional variation in the collagen fiber anisotropy and water content produces variation in the pattern of cartilage T2 values. This structural organization provides a well-recognized pattern of signal variation in Mill T2-weighted images, where low signal is observed near bone, gradually increasing in signal intensity toward the articular surface.

We postulated that disruption of this signal variation may be an early change of OA before the presence of symptoms or radiographic changes. Loss of collagen matrix anisotropy, one of the earliest processes in OA [8], leads to focal elevation in cartilage water content, increased mobility of the extra-cellular water, and ultimately loss of the ability of cartilage to with stand repetitive compressive loading. While early degeneration of the collagen matrix produces an elevation in cartilage T2, further degradation of cartilage produces heterogeneity in T2 values, with regions of cartilage demonstrating foci of low T2 values [9].

Recently, other groups have demonstrated a change in texture metrics in populations of patients with increased OA risk factors supporting this idea. Increased heterogeneity in the spatial distribution of cartilage T2 values is also a characteristic of aging, likely reflecting senescent degradation of the collagen matrix [2, 10]. Because T2 can increase or decrease regionally in cartilage, the bulk T2 value, which represents an average of multiple cartilage voxels over a region of interest (ROI), may remain unchanged, even while the variation of cartilage T2 from voxel to voxel may increase substantially. There may be no simple image “signature” of the disease that can be easily visualized and interpreted. Evidence of early OA progression in cartilage may manifest by subtle changes in image texture that occur on multiple scales across the huge space of voxels in the T2 map. The use of automated statistical classification techniques is directly motivated by problems of this nature where the data is high-dimensional. Together, these suggest that evaluation of changes in the pattern of cartilage T2 with OA progression may be a more responsive and reliable measure of cartilage degeneration than the change in absolute T2 values.

### SUMMARY OF THE INVENTION

In this work, an MM-based automatic classifier is designed to predict changes due to OA years prior to both their symptomatic presentation and radiographic detection. 220 patients were selected from the Osteoarthritis Initiative (OAI) database, 89 healthy and 131 symptomatic, based on the change in total WOMAC score from baseline to three year follow-up. For each patient, at baseline, 725 image texture features were measured from the T2 map of the patella cartilage and the lateral and medial compartments of the femoral condyle. A support vector machine (SVM)-based linear discriminant function was trained to predict health status, as well as the affected knee compartment, at three years from baseline. Feature selection was integrated into the classifier training to drastically reduce the number of image (biomarker) features without sacrificing classification accuracy. When the most important 20 of these 725 image features are used the method achieved an accuracy of 80% with a sensitivity of 79.2% and specificity of 68.5%. Further, it was found that a dominant knee compartment determined the classification decision for most patients. With this method, one may localize and identify regions of arthritis and cartilage damage to the patient's joint.

We demonstrate that the signal texture index (STI) predicts disease progression prior to symptoms or radiographic signs of OA, and, in symptomatic individuals, the STI correlate with the pain and severity of osteoarthritis suggesting it is a sensitive measure of early OA on T2 Maps. Further, these observed changes localized to one knee compartment suggesting that early OA occurs in primarily one compartment. Additional studies are required to determine whether the STI can be used to predict disease progression after post traumatic OA in the knee or in different joints and demonstrate response to therapeutic progression. The proposed method has clinical application for early arthritis diagnosis and treatment, the development and study of surgical procedures for cartilage repair and preservation, and to help identify and follow study populations to support both epidemiological and drug studies.

We hypothesized that this regional signal heterogeneity on T2 maps can be used as an early imaging biomarker to predict OA progression in asymptomatic individuals and as sensitive measure of early signs of OA. Early degenerative changes in the structural organization and water content of collagen in OA would be expected to have a regional change in signal as measured on T2 maps. These changes in regional heterogeneity can be quantified by texture metrics. We have utilized the OAI to define a population of individuals with no symptoms or radiographic sings of OA that are known to have rapid symptomatic progression in three years and a comparison asymptomatic control population. Image features were extracted from both populations and compared using classification to quantify and compare signal heterogeneity. We demonstrate that signal texture index can predict OA progression prior to OA onset. Further, the texture image features that are associated with OA progression are localized in a dominant compartment that is highly correlated with the mechanical axis of the knee. Our approach is to effectively utilize the signal texture index as a marker of cartilage degeneration, quantitatively assessed by measured texture features.

### BRIEF DESCRIPTION OF DRAWINGS

Further features, objectives and advantages of this invention will be made clearer with the following detailed description made with reference to the accompanying drawings in which:

FIG. 1 is a schematic flowchart according to one embodiment of this invention;

FIG. 2A is a graph comparing signal texture index (STI) versus density as means for identifying early signs of OA on a T2 map;

FIG. 2B is a graph plotting trial data using an SVM classifier from twenty dimensions onto two dimensions;

FIG. 3 is a graph plotting true versus false positive rates with the invention, the diagonal line therein representing random guessing;

FIG. 4A is a graph plotting STI versus density with the first, second and third compartments indicated along with the SVM decision boundary;

FIG. 4B is a graph showing the average STI, by notched box plots, for each of the three compartments; and

FIG. 4C is a graph plotting for individuals with a dominant medial or lateral compartment, the varus or valgus mechanical axis alignment associated with an increased STI.

### DESCRIPTION OF PREFERRED EMBODIMENTS

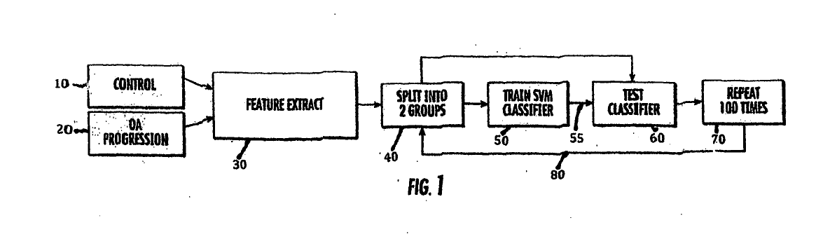

First, referring to the accompanying drawings, FIG. 1 shows a schematic representation of one experimental design according to this invention. Particularly therein, Control 10 and OA Progression 20 were fed as input to a Feature Extract 30. Output from that Feature Extract 30 was Split into 2 Groups 40 and used to Train an SVM Classifier 50, the SVM score output from which (Arrow 55) is a Signal Texture Metric. The latter gets forwarded to a Test Classifier 60 that went into a loop for repeating 100 times (Item 70). Output from the latter repeating (Arrow 80) looped back to the step of Splitting into 2 Groups (Item 40), one of which passes directly into Test Classifier 60.

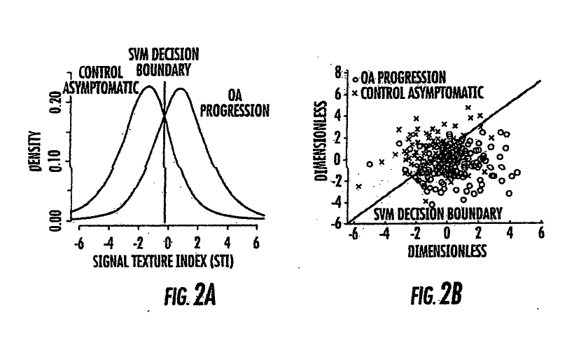

The Signal texture index identifies early signs of OA on T2 maps per accompanying FIG. 2. More particularly, FIG. 2A plotted histograms of the Signal Texture Index (STI) for the control and OA populations. A positive score therein corresponded to an OA decision, and a negative score a control decision. Accuracy was about 80%. Therein, the SVM decision boundary is indicated by the solid vertical line to the left of “0”. The results shown are the combined 1000 trials with an average number of 20 features needed to build the STI.

For FIG. 2B visualization, multidimensional scaling was used to project the data of one such trial using the SVM classifier from twenty dimensions onto two dimensions. Admittedly, that has some cost in representation fidelity. The axes are dimensionless, and represent a summation of the different image features used to determine the signal texture index (or “STI”).

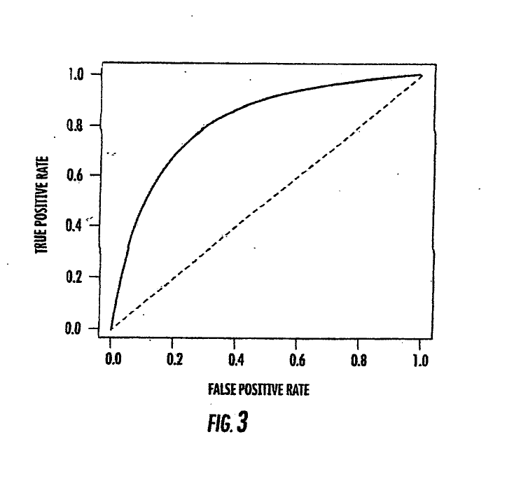

In FIG. 3, Receiver operating characteristic (or “ROC”) curve—The sensitivity is equivalent to the true positive rate, and specificity equivalent to true negative rate. The diagonal line therein represents the results of random guessing.

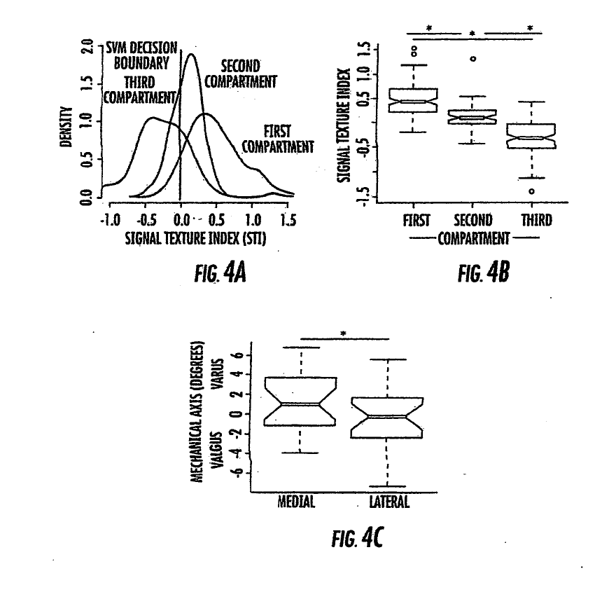

For the charts at FIG. 4, a signal texture index (STI) that indicates OA is associated primarily with one knee compartment. More particularly, FIG. 4A showed how the features that dominate OA decisions, for most subjects, come from primarily one knee compartment (medial, lateral, or patella). To demonstrate this, the aggregate STI “partial scores” were calculated for each knee compartment in each subject. A histogram for the compartment with largest partial score (“First”), second largest partial score (“Second”), and minimum partial score (labeled “Third”) across subjects as a function of Density where the area under each curve is unity. In that same FIG. 4A, the SVM decision score is shown as the vertical black line.

For FIG. 4B, the average STI in each compartment of 4A is statistically different. Notched box plots show the average STI for each of the three compartments. Finally, in FIG. 4C, the dominant compartment that predicted OA progression is strongly correlated with mechanical alignment. Notched box plot of individuals with a dominant medial or lateral compartment contribution to STI was compared as a function of the mechanical axis. Individuals with a varus alignment were associated with an increased STI in the medial compartment and vice versa for valgus alignment. The patella as the dominant compartment in the texture index was excluded. Notches note a 95% confidence interval of the mean. Negative mechanical axis values indicate valgus alignment. * p<0.05.

Population Cohort: Patients were selected from the OAI cohort. A total of 201 patients were selected, 89 control and 112 symptomatic. Specific inclusion criteria are as follows. Control subjects were selected from the control cohort defined as a low WOMAC score (<5) with low KL score that had no risk factors for OA progression. The OA rapid progression cohort were selected from the incidence cohort based on the initial criteria of a low WOMAC pain score less than 10 that had no radiographic signs of OA (KL<1) and that had a change in WOMAC pain score of >10. The incidence cohort did not have OA risk factors or symptomatic OA (risk factors: 1. previous knee surgery; 2. overweight as defined by ages cutoffs of 45-69 males>92.9 kg and females>77.1 kg; 3. previous knee injury defined by an injury of difficultly walking for at least one week; 4. family history in parent or sibling of total knee replacement; 5. Heberden's Nodes defined as self-report of bony enlargement of one or more enlargements of the distal interphalangeal joints in either hand; symptomatic OA: 1. Kellgren and Lawrence (KL) grade <2 on fixed flexion radiographs; 2. no frequent knee symptoms for at least one month during the past 12 months defined as “pain, aching, or stiffness in or around the knee on most days”). The incidence cohort did have risk factors for OA progression (OAI exclusion criteria included rheumatoid arthritis, bilateral total knee joint replacement, and a positive pregnancy test. Institutional review board approval had been obtained at all participating institutions in the OAI, and informed consent had been obtained by all participants in the study.

MR Image Acquisition: In the OAI cohort, three dimensional sagital DESS and T2 mapping images were acquired from the imaging database freely available by request [11]. Briefly, MRI of the knee joint was performed on a 3.0 T Siemens whole body MAGNETOM Trio 3T scanner (Siemens, Erlangen, Germany) using a standard extremity coil. For high-spatial-resolution 3D DESS imaging [12], a total of 160 sections were acquired with a field of view (FOV) of 14 cm (matrix 384×384) with an in-plane spatial resolution of 0.365×0.365 mm and a slice thickness of 0.7 mm with an acquisition time of 11 min. For sagittal 2D dual-echo fast spin echo (FSE) sequence for mapping T2 relaxation time, TR was 2700 ms and 7 echo images with TE ranging 10-80 ms were acquired with matrix of 384×384, in-plane resolution of 0.313×0.313 mm, FOV of 12 cm, acquisition time 12 min and slice thickness of 3 mm. OAI data sets used included the baseline imaging data set 0.E.1 and 0.C.2.

Plain Radiographic Assessment: Standard bilateral standing posterior-anterior fixed flexion knee radiographs were obtained at the baseline visit. Knees were positioned with a 20°-30° flexion and 10° internal rotation of the feet in a plexiglass frame (SynaFlexer, CCBR-Synarc, San Francisco, Calif., USA). Knee radiographs were graded using the using the Kellgren-Lawrence (KL) scoring system (Lawrence, 1957). The patello-femoral joint was not included in the KL score as the OAI protocol used the fixed flexion knee radiograph for KL scoring.

Standard bilateral, full length lower limb radiographs were obtained at the one-year clinical visit with knees fully extended and feet place six inches apart directly facing the film centered at the knees. Mechanical axis was measured using the standard technique of measuring the angle placed from the center of the femoral head to the medial tibial prominence to the midline of the ankle (McGory 2002, pub med id:12439260). OAI data sets used included the baseline and one year imaging data set 0.E.1 and 0.C.2.

Clinical Assessment: Clinical symptoms were assessed with the Western Ontario and McMaster Universities Osteoarthritis (WOMAC) questionnaire at the time of magnetic resonance screening (Bellamy, id 3068365). The OAI clinical data set 0.2.2 was used for data collection.

Registration: DESS and T2 images were registered using the Mattes mutual information metric. Registration software was built using the insight toolkit, a C++ open source image analysis library (www.itk.org). DESS images possess higher resolution and were transformed through three-dimensional space to preserve the voxel information on the fixed T2 image using a verser transform. Linear interpolation was used in sampling voxels on non-grid positions. A specialized gradient decent optimizer is used to define the transform parameters through successive iterations as the search space is large across 6 degrees of freedom. After the transform, the mutual information metric is used to assess the degree of alignment between the two images and the process is repeated until a maximum degree of overlap has been achieved (Urish 2013, pub med id: 23997865).

Segmentation: Segmentation was completed on DESS images. Segmentation of the femoral and patellar cartilage was completed using custom semi-automated software implementing a global active statistical shape model with a local active contour model. Gross inaccuracies in the segmentation could be corrected by a manual correction of the computer segmentation. Binary masks of the lateral and medial femoral condyle and patella were generated from the segmented images.

1. The lateral and medial masks were split into 5 sections for each individual. The patella region was treated as a single section. There were 11 regions of interest (ROI) per individual. The cartilage region for the lateral and medial compartments resembles an arc or semi-circle. In order to add an extra level of spatial localization ability to the feature set, the lateral and medial masks are divided into 5 sections each. Note that the medial and lateral masks are divided independently, so the section boundaries for the medial compartment are not necessarily the same as the ones for the lateral compartment. The automatic mask division algorithm is as follows: Find all segmented masks within the current patient's current compartment (medial or lateral).

2. Superimpose all segmented masks onto a single image.

3. Find θ, the angle of the “arc” of cartilage.

4. Divide θ by 5 to find θ_{S}, and then draw section boundaries at intervals of θ_{S}.

5. Starting on the far left and rotating counterclockwise, number the sections 1-5.

Note that the patella masks are usually much smaller than the lateral and medial masks, so they are not divided and are treated as a single section. Therefore, there are 5 medial sections, 5 lateral sections, and 1 patella section for a total of 11 sections. Each feature is measured independently in each section, so in general there are 11 instances of each feature. This is an arbitrary number and any number of regions of interest could be selected in operation of this invention. The specific segmentation technique used is not significant for our method and any type of segmentation method selected from the group of automatic, semi-automatic, and manual may be used.

T2 Maps: T2 maps were calculated from the Multi-Slice-Multi-Echo T2 images available in the OAI. Calculation of the T2 maps have been previously described (Smith and Mosher; Pubmed id 11436214). Briefly, the T2 maps are calculated on a voxel-by-voxel basis using a linear least squares fitting with CCHIPS/IDL software (Cincinnati Children's Hospital Image Processing Software/Interactive Data Language, (RSI, Boulder, Colo.). The MR T2 signal decay of cartilage is mono-exponential, and the signal intensity decay can be expressed as an exponential decay as a function of time for each voxel. Quantitative T2 maps can be visualized as a color-coded image using an ordinal rainbow scale.

Image Feature Extraction: Candidate features were calculated from each T2 map using the segmented binary masks region of interest using a matlab script (Mathworks, Natick, Mass.). Each feature was independently measured in each of the 11 sections on each knee. There were four main categories of features: histogram, grey level co-occurrence matrix (GLCM), grey level run length matrix (GLRL), and z-score. The numbers reported below are the totals from all 11 sections. A 32-bin histogram was used to calculate the mean, variance, entropy, and central moments. GLCM features were calculated from the grey level co-occurrence matrices at unit distance and angles 0, 45, 90, 135 degrees, and 90 degrees in the z direction. GLRL features were calculated from grey level run length matrices at angles 0 and 90 degrees. The Z-score was calculated for all voxels in each section. The mean value, variance, minimum value, maximum value, and range of values were then calculated (n=55). In each of the 11 sections, a total of 725 features were measured on each T2 map. All features were normalized to the range [−1,1].

Each mask is used to extract the appropriate voxels from the corresponding T2 map. As mentioned before, there are 5 medial sections, 5 lateral sections, and 1 patella section for a total of 11 sections that voxels can originate from. Features are measured independently for the voxels from each section, creating 11 instances of the same feature, each from a different location in the right knee.

Primarily, textural features were chosen to represent the images. This is because texture can measure statistical properties and spatial distribution of the image intensities [24]. This fact makes textural features very attractive for predicting OA status from the T2 map, and many of the initial features have been used in previous OA studies. The initial feature set can be split into four categories: 1) histogram; 2) gray level co-occurrence matrix (GLCM); 3) gray level run-length matrix (GLRL); 4) z-score.

All feature calculations are performed with custom-made Matlab functions. There are 725 total features in the initial feature set. In the following sections, let Iⱼ(u,v,w) be the intensity of the pixel at index (u,v,w) in section j and Sⱼ is the set of all voxel indices in section j.

Histogram Features: For each section, a 32-bin histogram is calculated from the intensity values of the T2 maps. The histogram in section j is calculated according to the equation:

$${p}_{i} ( x ) = \frac{ \begin{matrix}number of voxels in section j \\ with quantized gray level {q}_{x} in {S}_{j}\end{matrix}}{tota l number of voxels in {S}_{j}} , x = 0 , 1 , … , 31$$

Note that within section j, the histogram pⱼ actually does not depend on the spatial location of the voxels. This means that all histogram features do not depend on the location of the voxels within their specified section, but instead the histogram features measure the statistical properties of the voxel intensities. Even though there is no spatial dependency, histogram features are still often used in texture measurement. Also, some level of spatial information is included in this feature category since each feature is measured independently in each section, so each feature instance is localized to one specific location in the knee. The following equations define the histogram features:

$$Mean \text{:} {m}_{j} = {\sum }_{x = 0}^{31} {xp}_{j} ( x )$$

$$Variance \text{:} {σ}_{j}^{2} = {\sum }_{x = 0}^{31} {( x - {m}_{j} )}^{2} {p}_{j} ( x )$$

$$Dispersion \text{:} {\sum }_{x = 0}^{31} \vert x - {m}_{j} \vert {p}_{j} ( x )$$

$$Average Energy \text{:} {\sum }_{x = 0}^{31} {x}^{2} {p}_{j} ( x )$$

$$Energy \text{:} {\sum }_{x = 0}^{31} {[ {p}_{j} ( x ) ]}^{2}$$

$$Entropy \text{:} - {\sum }_{x = 0}^{31} {p}_{j} ( x ) {log}_{2} ( {p}_{j} ( x ) )$$

$$Skewness \text{:} {σ}_{j}^{- 3} {\sum }_{x = 0}^{31} {( x - {m}_{j} )}^{3} {p}_{j} ( x )$$

$$Kurtosis \text{:} {σ}_{j}^{- 4} ( {\sum }_{x = 0}^{31} {( x - {m}_{j} )}^{4} {p}_{j} ( x ) ) - 3$$

In addition to these features, the median, mode, minimum value, maximum value, and range of values is calculated from the histogram. An 8-bin histogram is also calculated for each section j and the occupancy of each bin is used as a feature. Also, the following two additional miscellaneous features are included in this category even though they are not calculated using the histogram.

$$Relative Size \text{:} \frac{number of voxels in {S}_{j}}{number of voxels in corresponding compartment}$$

$$L 2 norm \text{:} \sqrt{{\sum }_{( u , v , w ) ∈ {S}_{j}} {[ {I}_{j} ( u , v , w ) ]}^{2}}$$

Note that the “relative size” feature is not calculated for the patella section because there is only a single section in the patella compartment, which would make this feature equal to 1 for all patients.

Gray Level Co-Occurrence Matrix (GLCM): Histogram features alone cannot completely characterize texture since they do not measure the spatial characteristics of the cartilage region. The second-order histogram, called the gray level co-occurrence matrix (GLCM), is a common tool for measuring cartilage. In this study, the GLCM is calculated for a distance of 1 (the voxels must be immediate neighbors in the specified direction) and for direction θ=0°, 45°, 90°, 135°, and 90° in the z (third dimension) direction. Before the GLCM is calculated, the intensities are quantized down to 8 gray levels, which results in each GLCM being an 8×8 matrix.

If h_{j,θ}(x,y) is divided by the total number of neighboring pixels in section j, then the GLCM becomes an estimate of the joint probability f_{j,θ}(x,y). The following features are calculated from f_{j,θ}(x,y).

$$Angular Second Moment \text{:} {\sum }_{x = 0}^{7} {\sum }_{y = 0}^{7} {[ {f}_{j , θ} ( x , y ) ]}^{2}$$

$$Contrast \text{:} {\sum }_{x = 0}^{7} {\sum }_{y = 0}^{7} {( x - y )}^{2} {f}_{j , θ} ( x , y )$$

$$Absolute Value \text{:} {\sum }_{x = 0}^{7} {\sum }_{y = 0}^{7} \vert x - y \vert {f}_{j , θ} ( x , y )$$

$$Inverse Difference \text{:} {\sum }_{x = 0}^{7} {\sum }_{y = 0}^{7} \frac{{f}_{j , θ} ( x , y )}{1 + {( x - y )}^{2}}$$

$$GLCM Entropy \text{:} - {\sum }_{x = 0}^{7} {\sum }_{y = 0}^{7} {f}_{j , θ} ( x , y ) {log}_{2} {f}_{j , θ} ( x , y )$$

$$Correlation \text{:} {\sum }_{x = 0}^{7} {\sum }_{y = 0}^{7} \frac{{xyf}_{j , θ} ( x , y ) - {μ}_{u} {μ}_{v}}{{σ}_{u} {σ}_{v}}$$

Note that μᵤ, μᵥ, and σᵤ, σᵥ, are the means and standard deviations of the marginal distributions created by the row and column sums of the matrix, respectively. There are 5 different GLCMs (1 for each direction) calculated for each section, and since there are 11 sections this means that there are 55 total GLCMs calculated for a single patient. Each feature is measured independently for each GLCM.

Gray Level Run-Length Matrix (GLRL): The gray level run-length matrix (GLRL) is another typical tool used in texture analysis. The GLRL g_{j,θ}(x,y) is defined as the number of runs of length y in the direction θ consisting of points with gray level x in section j. Before the GLRL is calculated, the cartilage image intensities are quantized down to 8 gray levels. The GLRL is calculated for 0=0°,90°. The largest possible run length is the largest number of voxels that lie in direction θ, but the run lengths are quantized to 4 possible ranges. Let Pⱼ be the total number of voxels in section j, and let N_{j,θ} be the sum of all elements in g_{j,θ}. The following features are calculated from the GLRL g_{j,θ}(x,y).

$$Short Runs Emphasis \text{:} {\sum }_{x = 0}^{7} {\sum }_{y = 0}^{3} \frac{{g}_{j , θ} ( x , y )}{{y}^{2}} / {N}_{j , θ}$$

$$Long Runs Emphasis \text{:} {\sum }_{x = 0}^{7} {\sum }_{y = 0}^{3} {y}^{2} {g}_{j , θ} ( x , y ) / {N}_{j , θ}$$

$$Gray Level Nonuniformity \text{:} {\sum }_{x = 0}^{7} {( {\sum }_{y = 0}^{3} {g}_{j , θ} ( x , y ) )}^{2} / {N}_{j , θ}$$

$$Run Percentage \text{:} {\sum }_{x = 0}^{7} {\sum }_{y = 0}^{3} {g}_{j , θ} ( x , y ) / {P}_{j}$$

There are 2 different GLRLs per section, which means there are 22 total GLRLs calculated per patient. Each feature is calculated independently for each GLRL.

Z-Score: A special normalization procedure that produced features that had a significant correlation with OA damage can be defined as:

$$( u , v , w ) = \frac{{I}_{j} ( u , v , w ) - {μ}_{j , control}}{{σ}_{j , control}}$$

where μ_{j,control} and σ_{j,control} are the mean and standard deviation of section j for only the patients in the control group. This can be seen as normalization to “healthy” voxels, and therefore it may help the “symptomatic” voxels stand out more than normal. From the transformed intensities [symbol] , the features calculated are the mean, variance, minimum value, maximum value, and range of values. Similar to the histogram features, the z-score features do not have the ability to identify spatial characteristics of the voxels within section j. However, since the features are measured independently from each section, each feature instance is a localized measurement.

Feature Normalization: For each feature independently, each feature instance is normalized to the range [−1,1]. This is done because there might simultaneously be very large and very small feature values, which could cause numerical problems in the classifier training procedure

Classification, Feature Elimination, and Partial Sum Measurements: Given the features, we need to calculate a classification decision for each patient. The classification decision is reduced to a binary decision (healthy or symptomatic) for simplicity. In this case, each patient is treated as a 725-dimensional feature vector and the classifier maps this vector into a binary value. Since the feature space is very high-dimensional (especially considering there are only 210 patients in this 725-dimensional space), decreasing the dimensionality of this space could potentially lead to better generalization accuracy. Feature selection and classifier training are performed using a training set of patients, and then the generalization accuracy is estimated on a patient test set that is disjoint from the training set. This chapter is set-up as follows: the following section describes the support vector machine (SVM) classifier and the reason it was chosen, then the next section describes the feature selection algorithm margin-based feature elimination (MFE), and then the final section explains the structure of the feature selection and classification experiment.

Support vector machine: Support vector machine (SVM) training and testing were implemented using the LIBSVM Matlab interface. To assess the performance of the classifier, we randomly divided the entire cohort into 100 equal-sized training and test subsets with equal numbers of control and rapid progression individuals. In each of the 100 trials, the SVM classifier was trained to discriminate between control and rapid progression OA populations using all 725 features on the training set, and the accuracy of the classifier was measured on the independent test set. The confusion matrix was calculated after each trial. Margin based feature elimination (MFE) was used to eliminate redundant and uninformative candidate features. In the same trial, SVM training was coupled with MFE to identify a reduced set of essential features. The accuracy of the reduced feature set was tested on the test data set, and the confusion matrix was again determined (Arrow 80 in FIG. 1). After classification was completed and the signal texture index was calculated, the signal texture index of each compartment was determined. In each of the 100 trials, the partial weighted linear sum of the medial femoral condyle, lateral femoral condyle, and patella contribution to the each individuals overall SVM score was determined. Results were normalized based on the number of regions in each compartment (5 for the medial and lateral condyle, one for the patella), and averaged across the 100 separate trials. Any number of trial numbers, SVM or related classification methods, or methods to form the test and training sets on the available data sets could be used, and the method presented here is only an example of implementation.

As a specific example of implementation, each patient is represented as a data point in k-dimensional space, with k being the number of features. As mentioned previously, the number of initial features before the feature selection phase is 725 and the number of classes is 2. For now, assume these data points are linearly separable; in other words, the two classes can be separated using a single linear surface (hyperplane) of dimension k-1. This separating hyperplane essentially splits the k-dimensional space in two, with each subspace corresponding to one of the two classes. This hyperplane is known as the linear discriminant function (LDF), and in the case of SVM this hyperplane has the

form⁰=w^{T}x+b

where w is the kx1 SVM weight vector, x is the kx1 feature vector, and b is a scalar bias term. In this case, w is a vector normal to the hyperplane and |b|/∥w∥ is the perpendicular distance between the hyperplane and the origin where ∥w∥ is the Euclidean norm of the weight vector.

The choice of this separating hyperplane is not unique, and the choice of a particular hyperplane is the strength of SVM.

Since there are two classes, there are two class labels: −1 and +1, corresponding to healthy and symptomatic respectively. Let yᵢ be the class label for patient i and xᵢ be the feature vector for this patient. The SVM decision score for patient i is dᵢ and is written as

$${d}_{i} = {\sum }_{j = 0}^{k - 1} {w}_{j} {x}_{i , j} + b$$

and the actual classification decision for patient i is \hat{d}ᵢ=sgn(dᵢ)
where sgn is the signum function. For all training patients in a linearly separable data set, the following inequality holds:

yᵢdᵢ≧1

It can be shown that the patients from each class closest to the hyperplane are a perpendicular distance 1/∥w∥ away from it. This distance is called the margin of the hyperplane, and these patients are called the support vectors. The SVM procedure chooses a separating hyperplane such that the margin is maximized while satisfying the inequalities for every training instance. This can be stated formally as an optimization problem:
Minimize ∥w∥² subject to yᵢdᵢ≧1

The hyperplane is chosen to maximize margin because this hyperplane is proven to have better accuracy on unseen data points. Since we do not know the true distribution of data points in the feature space, the unseen data points may easily cross the separator and therefore be classified incorrectly. The separating hyperplane is chosen such that the margin between support vectors is maximized: the hyperplane with largest margin will typically account for the distribution of the classes better than other separators, and therefore it should perform better on unseen test data.

Other than maximizing margin between the classes and separating hyperplane, the strength of SVMs is the use of support vectors. In the derivation of the weight vector w and bias b, the support vectors are the only data points that affect w and b. This means that the classifier is uniquely determined by the choice of support vectors and only the support vectors. The number of model parameters in the classifier derivation depends on the number of support vectors. Therefore, unlike in other classifiers, the number of model parameters is not determined by the feature dimensionality and is instead bounded by the number of training instances, which causes the SVM to be more robust against overfitting.

Margin Based Feature Elimination: Feature selection is integrated into the classifier training process for this study. Selecting a subset of features from the initial feature set is necessary in order to eliminate redundant and non-informative features, and it is also possible to improve the generalization performance of the classification process [13].

Since margin-maximization is the goal of the SVM training procedure, a feature selection method was chosen that uses this margin as the criterion for removing features. This algorithm is called margin-based feature elimination (MFE). The goal of MFE is to maximize the SVM margin with each feature elimination step. This is a wrapper algorithm, meaning the trained classifier is used to determine the order of feature removal. In other words, classifier training is a part of the feature selection process, and the classifier is retrained many times to determine the usefulness of the features. MFE represents a computationally-efficient feature elimination algorithm that works together with the goal of the SVM training procedure and has been shown to perform well on standard datasets.

The MFE algorithm for linear SVMs has been described in detail [32]. A simplified version of the algorithm is explained: 1. Train a SVM using the current feature set. 2. Calculate the effect on the SVM decision function of removing each feature separately. 3. Remove the feature whose removal results in the largest SVM margin. 4. If linear separability is lost, stop. 5. Go to step 1, using the reduced feature set.

Note that in the version of MFE used in this study, the feature elimination is terminated when the training data becomes linearly nonseparable. MFE could be altered to continue removing features after this point, but this would require introducing slackness into the SVM training procedure. SVM training and MFE is simplified by not using slackness, and it was found experimentally that this method works well without considering slackness.

Statistics: Data is expressed as a mean±standard deviation, except where noted. Direct comparisons between two cell populations were made using an unpaired, two-tailed Student's t-test. Statistical significance was determined if P<0.05. Multiple group comparison's were made using two-way ANOVA, using the Student-Newman-Keuls pairwise comparison to determine significance levels. Conventions for box plot include the mean outlined by the box representing the 25% and 75% quantiles, whiskers representing the minimum and maximum value, outliers denoted with a circle, and notches representing the 95% confidence interval of the mean.

Receiver operating characteristic (ROC) analysis was performed on the entire set using standard techniques. The procedure and methods are discussed in detail using the thesis of Matthew Keffalas, The Pennsylvania State University, Electrical Engineering, Schreyer Honors College, 2010.

Classification Experiment: An example of implementing the entire method described above is as follows. The experiment is designed to estimate the performance of the combined SVM/MFE procedure. We must do this by using the 210 fully-preprocessed patients. According to the suggestions in [34], the patient set used to test the classifier performance must be completely disjoint from the patient set that is used to train the classifier and perform feature selection. Therefore we test the performance of the classification and feature selection methods by splitting the entire patient set into two equally-sized disjoint sets, training and test. The training set is formed by randomly sampling (without replacement) the full dataset. Each set has the same ratio of classes as the original dataset. The training set is used to select the sparse feature set and train the SVM, and the test set is used to estimate the generalization accuracy of the trained classifier and the selected feature set. This procedure is repeated for 100 trials using 100 Different training sets (and therefore 100 different test sets), and the final performance of these methods are estimated by averaging the results from each trial.

We hypothesized that T2 map signal heterogeneity could accurately prognosticate OA progression. To test this hypothesis, we used the OAI to identify and compare the texture metrics of two populations of T2 maps: an asymptomatic control and a rapid OA progression population. The asymptomatic group was collected from the OAI control cohort (n=89). The rapid progression population was collected from the incidence cohort (n=112). At the initial time point, the population was asymptomatic (WOMAC<10) and had no radiographic signs of OA (KL≦2). At the 3 year time point, this population experienced a WOMAC change (greater than 10), signifying both a large and rapid progression of symptoms. These populations were comparable in regards to age, sex, and BMI. As expected by cohort definitions between the control and incidence cohorts, the asymptomatic population did have lower WOMAC and KL scores than the rapidly progression population.

To assess signal heterogeneity, we quantified signal heterogeneity using a series of texture metrics on each of these populations. Asymptomatic and rapid progression populations based on baseline T2 map image features that described texture. Images were segmented and registered so that image texture features could be extracted. DESS images were used for segmentation because of the increased contrast at cartilage-soft tissue and cartilage-bone interfaces. Multimodality registration was used to align DESS and T2 sequences so that the segmentation masks (subdivided into ROI) could be used to measure a range of histogram and texture based image features. We chose as candidate features some well-known descriptors of image texture (local entropy, variance, cross-correlation, run-lengths, histogram based) and integrated a feature reduction step within the classifier training. An image classifier, SVM, was used to develop a model to predict OA, with classifier training and testing via a series of cross-validation experiments, dividing the subpopulations into training and test subsets. Margin based feature elimination was used to eliminate redundant and uninformative features, sacrificing minimal accuracy in order to simplify the model and to identify image feature biomarkers. The classifier design “wraps” MFE around SVM training, removing one feature at a time. The classifier found an appropriate hyper-plane in image-feature space that separated these populations. The SVM score is the distance from this plane, and can be described as a signal texture metric (Arrow 55 in FIG. 1).

Three separate cases of classifier accuracy were analyzed. First, the accuracy of using the entire set of all 725 features before feature elimination was measured. The average accuracy of the classifier was greater than 80%, corresponding to an average sensitivity of 82.0±5.4% and an average specificity of 75.0±7.3%. Second, MFE was used to remove redundant and uninformative features significantly reducing the feature space. An average of only 20 of the 725 features was needed to maintain a comparable level of accuracy. The average accuracy of the system with MFE feature selection was 76.1±7.2%, with average sensitivity of 79.1±6.7% and average specificity of 70.0±7.7%. Finally, in each of these trials by design, a new separate set of features was selected. If feature reduction was performed on the entire trial set simultaneously, a single set of features for the signal texture index were defined. At a sacrifice of some bias to obtain the single feature set, accuracy was 80% with a sensitivity of 83% and a specificity of 77%. The remainder of the discussion will focus on the second case as it presents the most unbiased classifier accuracy and quantification of signal texture index.

After feature reduction, the STI had good separation of the asymptomatic and OA populations. By design, the classifier sets a STI value of zero as the decision boundary so that any positive value is determined to be OA progression and any negative value a control decision is made (FIG. 2A). One of the trials with similar accuracy (76.6%) to the entire set of trial corresponds with good separation of the two populations (FIG. 2B). ROC analysis showed excellent classifier performance, and tradeoffs between specificity and sensitivity as a function of the SVM decision boundary (FIG. 3). This invention can be used to prognosticate and/or diagnose cartilage damage, arthritis, or the general state of cartilage health. This demonstrates the minimum accuracy of the invention. Small modifications will improve the accuracy.

The image texture features that predict rapid progression of OA for most individuals are primarily located in one of the three knee compartments. The STI is calculated from a weighted sum of image feature measurements from the lateral and medial compartment and the patella. By separately considering the features from each compartment (lateral, medial, patella) and finding the partial sum for each section, the effective contribution from each compartment to the overall decision can be determined. The rapid progression population was considered separately in this analysis. The contribution of features in each compartment to the overall signal texture index shows substantial separation between each compartment (FIG. 4A) and the mean of each of these compartments are statistically different (FIG. 4B). This suggests that, for most subjects, a single knee compartment plays a dominant role in rapid progression to symptomatic OA.

To test the observation that the signal texture index from one compartment plays a dominant role in OA progression, we isolated the medial and lateral sub-populations from the dominant compartment and compared the compartment to the mechanical axis from standing full limb length radiographs. Individuals with a dominant compartment on the medial condyle were highly correlated with valgus alignment, and individuals associated with a dominant compartment on the lateral condyle were associated with a varus alignment. A comparison of these two populations demonstrated the differences were statistically different as measured by the student's t-test. At a minimum, the dominant compartment's location is highly correlated with mechanical axis (FIG. 4C).

For symptomatic patients that are correctly classified, the most positive partial sum amongst the three sections contributes most to the correct decision. It can thus be inferred that the section with this partial sum is likely the one undergoing the most OA changes. Moreover, a significant disparity between the largest and second largest partial sums for an individual patient suggests OA changes may only be occurring in the knee section with the largest partial sum. Thus, this invention may be used to localize cartilage damage, the progression of disease, or identify area of the joint where healthy cartilage resides.

This invention had been demonstrated to be used on T2 maps for OA symptomatic prognostication but the demonstration is equally valid on a number of other instances. Any MR sequence including but not limited to dGERMIC (i.e, delayed gadolinium-enhanced magnetic resonance imaging of cartilage), T1, T1 rho, and T2 sequences could be substituted or combined with T2 maps. Texture and feature analysis was used to prognosticate but could be used as a diagnostic test for symptomatic progression or differentiate different stages of disease progression. Further, the same invention can be applied to predicting morphologic changes in articular cartilage including but not limited to changes in cartilage volume, area, or thickness and bone, synovial, inflammatory tissue morphometry. The invention is not specific to the disease of OA but any type of pathologic process effecting human or animal cartilage including but not limited to rheumatoid arthritis, post-traumatic arthritis, cartilage trauma, osteochondritis dissecans, or the general state of cartilage health. Also, the invention can be applied to any joint in the body including but not limited to hip, shoulder, elbow, wrist, and ankle. The specific steps used in this invention can be altered in length, method employed, order, or omission.

A challenge in classification is the “curse of dimensionality”. There is a relative paucity of available training samples compared to the large dimensionality of the image feature space and to the number of parameters in the classifier model. This implies that the classifier has a tendency to overfit the data which can degrade the accuracy of the model. To avoid this problem, we applied a linear discriminant function classifier, SVM, to maximize the margin between these two populations. In this sense, the SVM maximizes the separation of the two classes. For an SVM, unlike a standard linear discriminate function, the number of model parameters is bounded by the number of training samples, rather than being controlled by the feature dimensionality. Since the number of samples is typically the much smaller number, in this way the SVM mitigates potential overfitting. SVM is not a unique solution to this process. Any linear or non-linear classifier and method of feature reduction can be applied for use in this invention. An emphasis was placed on defining OA by symptomatic progression. OA could be defined by symptoms, biomarkers, imaging criteria, or any other definition.

In addition to overcoming the problem of the non-linear response of T2 to cartilage degradation, this approach removes systematic bias. Differences in methodology or instrumentation used in the T2 measurement can lead to differences in the magnitude of T2 values. Since texture analysis compares spatial differences in T2 values between neighboring pixels rather than the absolute T2 values, it effectively uses an internal calibration standard to remove systematic bias. This helps eliminate the variation observed in a sequence as a function of the operator, machine, and location.

There are distinct differences between results discussed here and those reported by Dam [14]. In Dam's work, they make distinctions between non-progressers and early progressors. Dam uses the definition of early progressors based on radiographic progression. For example, at the end of the time period non-progressors have no change in radiographic findings and early-progressors have an increased change in radiographic findings. Our method presented above and as demonstrated by our results can perform the more difficult task of identifying patients that will have symptomatic progression with no radiographic progression or evidence. For example, Dam's method has the potential to misclassify an individual as an arthritis non-progressor based on no predicted change in their radiographs when they may have had arthritis progression based on symptoms. Our method would have improved accuracy and prognostication by correctly classifying these individuals.

From this perspective our method identifies an even earlier type of progression in the disease process that is more challenging to detect than the Dam group. A large disadvantage of radiographs is that radiographs are a poor method to detect early arthritis changes (CITE). Individuals may develop artrhitis symptoms without having radiographic evidence of artrhitis (CITE). Using Dam's definition [14], this group would be entirely missed by his analysis and considered non-progressors. Using our definition and method, this group would not be miss-classified and correctly considered part of the progression. This allows are method to be more sensitive and specific for early arthritis progression than Dam's method. Further, Dam demonstrates that there are differences between progressors and non-progressors but does not actually demonstrate that it can be used as a prognostic tool [14]. Our results presented above, demonstrate its prognostic accuracy.

Footnoted References above:
1 David-Vaudey, E., et al., T2 relaxation time measurements in osteoarthritis. Magn Reson Imaging, 2004. 22(5): p. 673-82.
2. Mosher, T. J. and B. J. Dardzinski, Cartilage MRI T2 relaxation time mapping: overview and applications. Semin Musculoskelet Radiol, 2004. 8(4): p. 355-68.
3. Mosher, T. J., et al., MR imaging and T2 mapping of femoral cartilage: in vivo determination of the magic angle effect. AJR Am J Roentgenol, 2001. 177(3): p. 665-9.
4. Wheaton, A. J., et al., Correlation of T1rho with fixed charge density in cartilage. J Magn Reson Imaging, 2004. 20(3): p. 519-25.
5. Burstein, D. and M. L. Gray, Is MRI fulfilling its promise for molecular imaging of cartilage in arthritis? Osteoarthritis Cartilage, 2006. 14(11): p. 1087-90.
6. Xia, Y., J. B. Moody, and H. Alhadlaq, Orientational dependence of T2 relaxation in articular cartilage: A microscopic MRI (microMRI) study. Magn Reson Med, 2002. 48(3): p. 460-9.
7 Harrison, M. M., et al., Patterns of knee arthrosis and patellar subluxation. Clin Orthop Relat Res, 1994(309): p. 56-63.
8. Maroudas, A. I., Balance between swelling pressure and collagen tension in normal and degenerate cartilage. Nature, 1976. 260(5554): p. 808-9.
9. Yulish, B. S., et al., Juvenile rheumatoid arthritis: assessment with MR imaging. Radiology, 1987. 165(1): p. 149-52.
10. Mosher, T. J., B. J. Dardzinski, and M. B. Smith, Human articular cartilage: influence of aging and early symptomatic degeneration on the spatial variation of T2-preliminary findings at 3 T. Radiology, 2000. 214(1): p. 259-66.
11. Peterfy, C. G., E. Schneider, and M. Nevitt, The osteoarthritis initiative: report on the design rationale for the magnetic resonance imaging protocol for the knee. Osteoarthritis Cartilage, 2008. 16(12): p. 1433-41.
12. Eckstein, F., et al., Double echo steady state magnetic resonance imaging of knee articular cartilage at 3 Tesla: a pilot study for the Osteoarthritis Initiative. Ann Rheum Dis, 2006. 65(4): p. 433-41.
13. Aksu, Y., et al., Margin-maximizing feature elimination methods for linear and nonlinear kernel support vector machines. IEEE Trans On Neural Networks, 2010. 21: p. 701-717.
14. Dam, E. B. K., DK), Qazi, Arish (Kobenhavn k, DK), Karsdal, Morten (Kobenhavn k, DK), Petterson, Paola C. (Koge, DK), Nielsen, Mads (Dragor, DK), Christiansen, Claus (Morcote, CH), Pathology indicating measure related to cartilage structure and automatic quantification thereof. 2010, Nordic Bioscience Imaging A/S (Herlev, DK): United States.

What is claimed is:

1. A method for detecting a type of symptomatic osteoarthritis to a joint or cartilage of a human patient who is otherwise asymptomatic, said method comprising:
(a) taking magnetic resonance (MR) sequence maps of one or more regions of cartilage in a joint of the patient;
(b) submitting said MR sequence maps to a classifier for performing a feature reduction that calculates which redundant or unnecessary features may be removed;
(c) eliminating said redundant or unnecessary features from said MR sequence maps to form a minimum grouping of image features for the patient;
(d) calculating from said minimum grouping of image features a signal texture index (STI) value;
(e) comparing the STI value for the patient against at least two population databases, a first database including individuals known to develop osteoarthritis at a first time point and a second database including individuals known not to develop osteoarthritis at a later time point;
(f) prognosticating from the STI value, a likelihood of the patient developing osteoarthritis; and
(g) treating the patient based on prognosticating step (f).

2. The method of claim 1 which can be used to prognosticate, from the STI value, a likelihood of the patient developing arthritis or damage.

3. The method of claim 1 wherein step (f) includes prognosticating the likelihood that osteoarthritis will progress or regress in the patient.

4. The method of claim 3 wherein step (f) includes prognosticating the rate of progression or regression of osteoarthritis in the patient.

5. The method of claim 1 wherein the joint or cartilage is in the patient's knee, ankle, hip, shoulder, elbow, wrist or spine.

6. The method of claim 5 wherein the joint or cartilage is from the patient's knee, and the magnetic resonance (MR) sequences for that knee are taken for the patient's patella, medial and lateral compartments.

7. The method of claim 6 wherein the image features used to calculate the STI are taken mostly from a dominant compartment of the patient's knee, said dominant compartment selected from the patient's patella, medial or lateral compartment.

8. The method of claim 1 wherein the classifier from step (b) is selected from the group consisting of a linear classifier, a non-linear classifier, a regression framework and a neural network.

9. The method of claim 1 wherein step (d) includes using one or more histogram measures selected from the group consisting of: mean, standard deviation, variance, dispersion, average energy, energy, skewness and kurtosis.

10. The method of claim 1 wherein step (d) includes using one or more measures selected from the group consisting of: gray level co-occurrence matrix (GLCM), gray level run length (GLRL), and Z-scores.

11. A method for prognosticating and treating osteoarthritis of a patient's knee or hip, said method comprising:
(a) taking magnetic resonance (MR) sequence maps of one or more regions of the patient's knee or hip;
(b) submitting said MR sequence maps to a classifier for performing a feature reduction that calculates which features may be removed;
(c) eliminating said features from said MR sequence maps to form a minimum grouping of image features for the patient;
(d) calculating from said minimum grouping of image features a signal texture index (STI) value for the patient;
(e) comparing the patient's STI value against a plurality of population databases, at least one database for individuals known to have already developed osteoarthritis and a second database for individuals known to have not yet developed osteoarthritis;
(f) prognosticating from the STI value, a likelihood of the patient developing osteoarthritis; and
(g) treating the patient based on prognosticating step (f).

12. The method of claim 11 wherein step (f) includes prognosticating the likelihood that osteoarthritis will progress or regress in the patient.

13. The method of claim 12 wherein step (a) includes taking magnetic resonance (MR) sequences for the patient's patella, medial and lateral compartments.

14. The method of claim 13 wherein the image features used to calculate the STI are taken mostly from a dominant compartment of the patient's knee.

15. The method of claim 11 wherein the classifier from step (b) is selected from the group consisting of a linear classifier, a non-linear classifier, a regression framework and a neural network.

16. The method of claim 11 wherein step (d) includes using one or more histogram measures selected from the group consisting of: mean, standard deviation, variance, dispersion, average energy, energy, skewness and kurtosis.

17. The method of claim 11 wherein step (d) includes using one or more measures selected from the group consisting of: gray level co-occurrence matrix (GLCM), gray level run length (GLRL), and Z-scores.
